(19) United States
(12) Patent Application Publication
(10) Pub. No.: US 2006/0018787 A1
(43) Pub. Date: Jan. 26, 2006

(54) Synchronized electronic smell emission method and device for television programs, movies and other programs

(76) Inventor: Yixin Guo, Ronkonkoma, NY (US)

Correspondence Address: Yixin Guo, 256 Holbrook Avenue, Ronkonkoma, NY, 11779, US

(21) Appl. No.: 10/962,762

(22) Filed: Oct. 13, 2004

Related U.S. Application Data
(60) Provisional application No. 60/589,553, filed on Jul. 21, 2004.

(51) Int. Cl. A61L 9/04; A61L 9/12

(52) U.S. Cl. 422/5; 422/124; 422/305; 422/306

(57) ABSTRACT

It is a kind of method and device relating to the synchronized electronic smell emission for television programs, movies and other programs. This invention shows that according to the corresponding video and audio contents in the television programs, movies, electronic games and computer software, a program card that contains synchronized smell information is pre-produced. The program card operates a group of control valves through control circuit board to open or close a corresponding group to emit a same type of smell from the smell containers which contain different types of smell to correspond synchronously to the video and audio contents in the television programs, movies, electronic games and computer software and the emitted smells are then blown into the air by the fan. In addition to the plentiful video and audio effects provided in the existing television programs, movies, electronic games and computer software, this invention provides a smell effect which corresponds synchronously to the contents of a picture by adopting a simple method which is not associated directly with the current video and audio signals so that viewers can actually sense the three-D effects of the sense of sight as well as the sense of hearing and the sense of smell in the viewing and entertaining process, and accordingly the enjoyment quality of the television programs, movies, electronic games and computer software as well as the entertainment effect of the electronic games will be further enhanced.

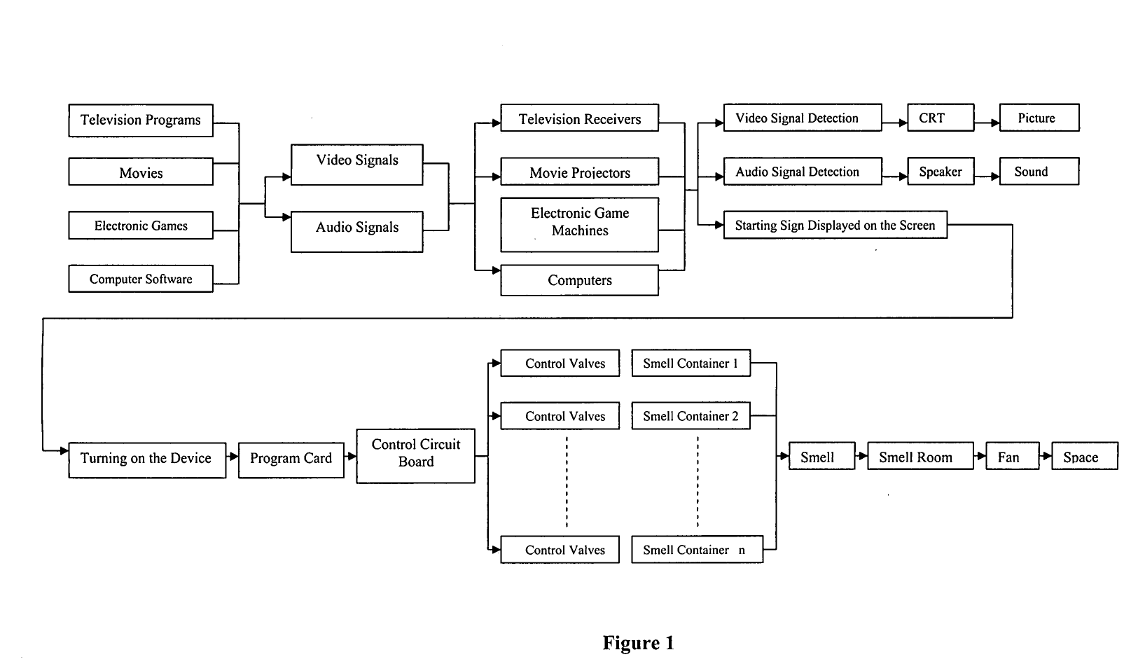

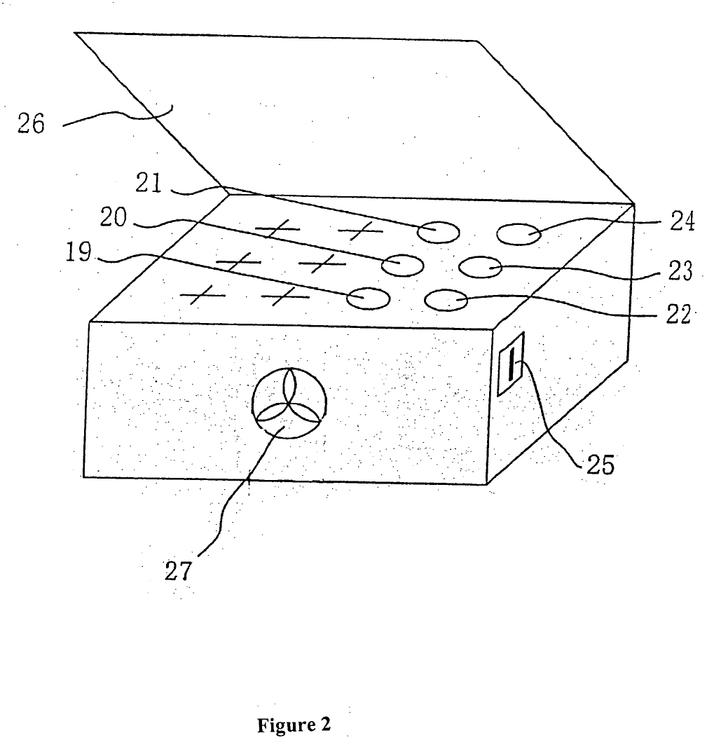

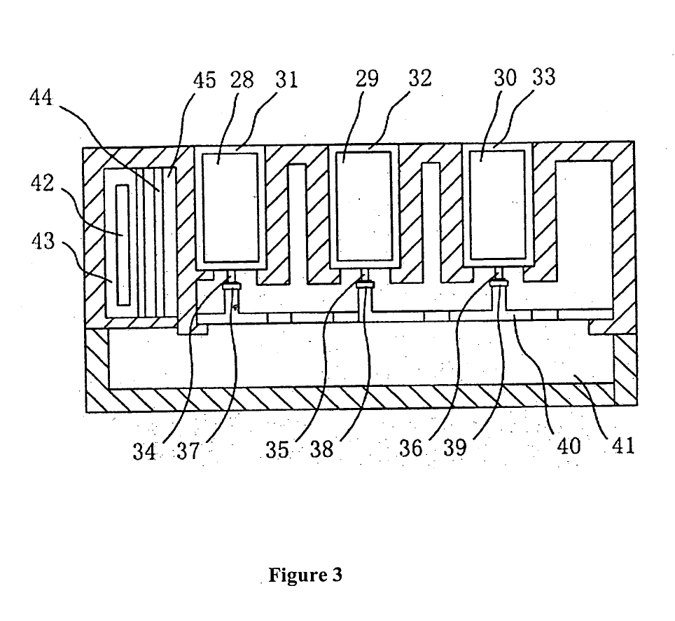

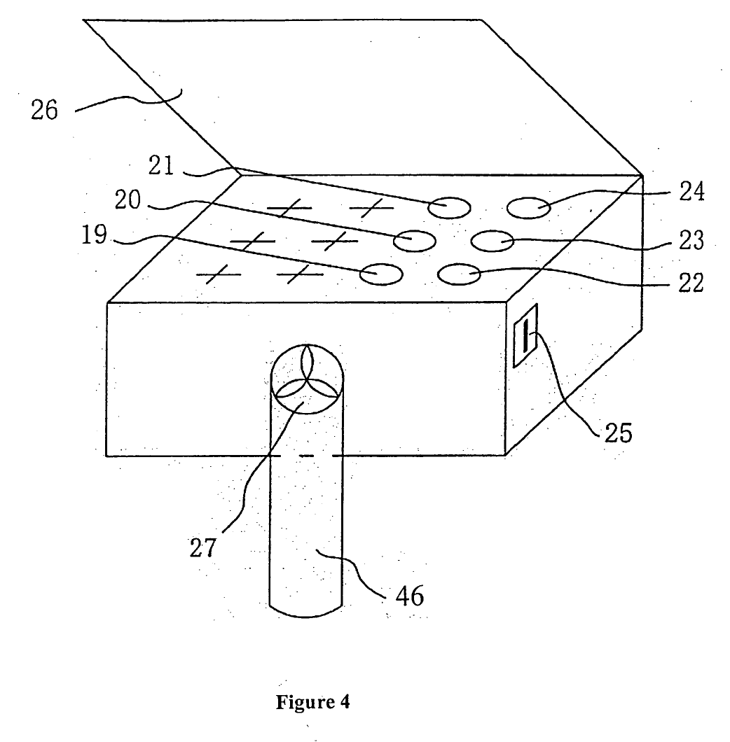

### FIELD OF THE INVENTION

This invention relates to a kind of smell information technology for the television receivers, movie projectors, electronic game machines and computers and more particularly to the method and device in which the smell signals are transmitted synchronously corresponding to the video and audio signals in the television programs, movies, electronic games and computer software. This kind of smell signal is not mixed with video and audio signals, but is solely transmitted from the synchronized electronic smell emission device which is controlled by the program card to start the electromagnetic valves through the control circuit board and open the corresponding smell container openings to emit the smells that correspond to the pictures on the screens of the television receivers, movie projectors, electronic game machines and computers.

### BACKGROUND OF THE INVENTION

Nowadays, television programs, movies, electronic games and computer software have become an indispensable part in people's lives. These programs not only bring the pleasure to the people, but also provide a great deal of knowledge and information. The conventional television programs, movies, electronic games and computer software have been focusing on developing the effect of the sense of sight and the sense of hearing. However, in addition to the sense of sight and the sense of hearing, people also need to be satisfied with the sense of feeling as well as the sense of smell and the sense of taste when receiving the information. If the ways to provide the information through television programs, movies, electronic games and computer software are confined to the sense of sight and the sense of hearing only, people will be inevitably limited to enjoy these cultural and artistic effects.

As everyone knows, there have been no any cultural and artistic compositions of television programs, movies, electronic games and computer software in the existing technology that offer the effect of the sense of smell at the same time when the effect of plentiful sense of sight and sense of hearing are offered. The major difficulty is that the smell is a sort of chemical compound and its element composition is too complicated to be compounded immediately with several other single elements under general circumstances. Secondly, very few sensors can convert the smell into an electrical signal at the present time. In terms of the input and output signals in the movies and television programs, there is no any device that is able to receive or emit the smell signal. On the other hand, even though people have realized the function and the effect of the sense of smell for a long time, the appropriate method of how to apply the sense of smell into movies and television programs to actually constitute the three-D effects of the sense of sight as well as the sense of hearing and the sense of smell has not been discovered in the long time practice.

### SUMMARY OF THE INVENTION

This invention provides a kind of method and device relating to the synchronized electronic smell emission for television programs, movies and other programs. In addition to the plentiful video and audio effects provided in the existing television programs, movies, electronic games and computer software, it is the object of this invention to provide synchronously a smell effect corresponding to the contents on a picture by adopting a simple method which is not associated directly with the current video and audio signals so that viewers can actually sense the three-D effects of the sense of sight as well as the sense of hearing and the sense of smell in the viewing and entertaining process, and accordingly the enjoyment quality of the television programs, movies and computer software as well as the entertainment effect of the electronic games will be further enhanced.

In order to accomplish the above object, the technical plan adopted by the present invention of the synchronized electronic smell emission method for television programs, movies and other programs is that a program card that contains synchronized smell information is pre-produced according to the corresponding video and audio contents in the television programs, movies, electronic games and computer software to operate a group of control valves through control circuit board and open or close a corresponding group to emit a same type of smell from the smell containers which contain different types of smell to correspond synchronously to the video and audio contents in the television programs, movies, electronic games and computer software, and the emitted smells are then blown into the air by the fan.

The following is the explanation of the relevant contents in the above-mentioned invention:
1. Said smells in the above plan include the scent of flowers, perfume smell, coffee smell, smell of a fried dish, fruit smell, smell of sourness, spicy smell, stinking smell, fishy smell, smoke and fire smells and ammunition smell.
2. In the above invention, in order to enhance the smell emission effects, the emitted smells can enter a smell room first and are then blown into the air by the fan.
3. In the above invention, the smells in each said smell container can be the following types: the smells in each smell container are composed of the compressed gas of each different type of smell and the smells are emitted from the smell containers when the control valves are opened.
4. In the above invention, the corresponding smell type, emission time, time length and intensity level are shown through the choice of different smell containers and the container opening time, time length and the opening size at the time of smell emission. This type of expression is programmed by the program card according to the contents required.
5. In the above invention, a signal of starting the smell emission is displayed on the screen when the television programs, movies, electronic games and computer software have just started in order to ensure that the smell emission corresponds synchronously to the contents of the picture and the sound.
6. In the above invention, methods of digital-digital control, digital-analog control and analog-digital control are adopted in the whole smell emission control process ranging from the program cards to control valves.
7. In the above invention, the emitted smells, in terms of large locations such as the movie theaters, are discharged through the suction port of the air conditioning or ventilation system or a special discharge port is separately set up.

In order to accomplish the above object, the technical plan adopted by the present invention of the synchronized electronic smell emission device for television programs, movies and other programs is that a group of smell containers are installed in the device and each smell container contains different type of smell. The smell containers are placed separately at each disposition space or at the corresponding location. There is a smell room in the device in where the inlet which connects each container opening is installed. The control valve is respectively installed at each container opening. The outlet which connects the exterior space is also installed in the smell room and a fan is installed at the outlet or in the smell room. The device is also equipped with a program card and the control circuit board. The control circuit board is either a control circuit or a control unit that carries a CPU. Its input port receives the control signal and its output port is connected respectively to the control end of each control valve. The program card is a unit that transmits the control signal. This unit comprises a smell signal program or a circuit in which a same type of smell corresponds synchronously to the video and audio contents of the television programs, movies, electronic games and computer software. The unit is connected to the control circuit board through a connection port.

The following is the explanation of the relevant contents and changes in the above-mentioned technical plan:

In the above plan, the said control valves adopt the electromagnetic valves, hydraulic control components, pneumatic components or servo control components. The choice of the control valves depends on the requirement of the smell emission. For example, the best effect can be obtained if the servo control components are used when controlling the opening size of the smell container is needed.

The working principle of this invention is: the smells are synchronously emitted corresponding to the contents of the picture and sound that appear on the screens of the television receivers, movie projectors, electronic game machines and computers. In accordance with this invention, each smell container in the smell emission device is controlled by a special program card. It is an independent system which is not associated electronically with the television receivers, movie projectors, electronic game machines and computers, and there is no need to change the existing apparatus like television receivers, movie projectors, electronic game machines and computer hardware as well as the video and audio signals in the television programs, movies, electronic games and computer software. Therefore, this is a practicable and workable invention.

Due to the adoption of the above technical plan, this invention has the following advantages and effects compared with the existing technology:
1. Since the smell information in the present invention synchronously matches the original video and audio information in the television programs, movies, electronic games and computer software, the sense of sight as well as the sense of hearing and the sense of smell constitute the three-D integration and the enjoyment effect will be greatly improved.
2. Since the synchronized smell information is added in the present invention to the original video and audio information in the television programs, movies, electronic games and computer software, the production and market of the television programs, movies, electronic games and computer software will be developed towards a new field.
3. Since the present invention provides the information technology of the synchronized electronic smell emission device that is associated with the television receivers, movie projectors, electronic game machines and computers, the technical research, production and market of the television receivers, movie projectors, electronic game machines and computers and the synchronized electronic smell emission device will be developed towards a new field.
4. It is a practicable and workable invention since there is no need to change the existing apparatus like television receivers, movie projectors, electronic game machines and computer hardware, and the sense of smell is added.

### BRIEF DESCRIPTION OF THE DRAWINGS

DRAWING 1 is the block diagram of the present invention associated with the existing television programs, movies, electronic games and computer software.

DRAWING 2 is the solid diagram of the application example of the synchronized electronic smell emission device.

DRAWING 3 is the A-A sectional view of DRAWING 2.

DRAWING 4 is the solid diagram of the synchronized electronic smell emission device used in the movie theaters and other large locations.

In the above drawings, 19 to 24: disposition space of smell containers. 25: card plugging jack. 26: box cover. 27: fan. 28 to 30: smell disposition space. 31 to 33: smell containers. 34 to 36: smell container openings. 37 to 39: electromagnetic valves. 40: baffle. 41: smell room. 42: program card. 43: program card space. 44: control circuit board. 45: control circuit board installation space. 46: smell channel.

### DETAIL DESCRIPTION

The following is the further description of the present invention in conjunction with the accompanying drawings and the application examples:

A kind of synchronized electronic smell emission method for television programs, movies and other programs which correspond to each television program, movie, electronic game and computer software so as to program the smell type, smell intensity, the starting time and time length according to the contents of the picture and the sound and input all the information into the program card of the synchronized electronic smell emission device. The emission starting time and what kind of smell should be emitted as well as the time to stop the smell emission and the smell intensity should be programmed according to the contents in the television programs, movies, electronic games and computer software when the contents of the smell emission are designed and the corresponding setting should be written into the program and inputted into the program card which can be assigned to each television program, movie, electronic game and computer software. When viewing a program, the program card corresponding to this program should be plugged into the input jack on the synchronized electronic smell emission device which is connected to the interior control circuit board through the interface circuit. When the television programs, movies, electronic games and computer software have just started, a signal of starting the smell emission should be displayed briefly on the comer of the screen. At this time, the user should turn on the control switch to start the synchronized electronic smell emission device. Then the program card, as programmed, would send out a signal at a certain time to operate an electromagnetic valve through control circuit board to open the corresponding smell container and emit the same type of smell with appropriate smell intensity corresponding to the contents of the picture and the sound of the television programs, movies, electronic games and computer software and then the smell enters into the smell room which has the buffering function and is then blown into the air by the fan as shown in Drawing 1. After a period of time, the electromagnetic valve and fan are turned off through control circuit board to stop the smell container from emitting the corresponding smell following the signal sent out from the program card.

The smells in each said smell container are composed of compressed gas of different types of smell. The smells are emitted from the smell containers when the control valves are opened. The smells in each smell container can be also composed of liquids of different smell types. When the control valves are opened, the smell liquids flown out or emitted are vaporized into gas by electrical heating in the smell room and are then blown into the air by the fan. The said smells include the scent of flowers, perfume smell, coffee smell, smell of a fried dish, fruit smell, smell of sourness, spicy smell, stinking smell, fishy smell, smoke and fire smells and ammunition smell. The corresponding smell type and emission time, time length and intensity level are shown through the choice of different smell containers and the container opening time, time length and the opening size at the time of the smell emission.

Referring to Drawing 1, there is shown the working principle of this invention associated with the television programs, movies, electronic games and computer software. When the television programs, movies, electronic games and computer software which contain video and audio signals are playing, the television receivers, movie projectors, electronic game machines or computers would detect such video and audio signals. The video signals are transmitted to CRT to show the pictures and the audio signals are transmitted to the speaker to hear the sound. In order to synchronize the smell signal with the video and audio signals, a signal of starting the smell emission should be displayed briefly on the corner of the screen when the programs have just started. At this time, the user should turn on the switch immediately or use the remote controller to start the synchronized electronic smell emission device which would, as programmed, emit different smells at different times corresponding to the contents of the picture.

Application Example: referring to Drawing 2 to 3, there is shown a kind of synchronized electronic smell emission box for television programs, movies and other programs which comprise the space to dispose several smell containers 19 to 24, smell containers 31 to 33, electromagnetic valves 37 to 39, control circuit board 44, fan 27, program card 42, smell room 41 as well as the switch and remote controller.

In this application, the space to dispose 24 or 36 or more smell containers can be arranged. As shown in Drawing 2, only part of the smell container spaces 19 to 24 are shown in this drawing. The different types of smell are stored in the smell storage space 28 to 30 in the smell container. Referring to Drawing 3, the smell containers 31 to 33 are disposed respectively in the smell container spaces 22 to 24. The smell containers can be used for one time only or for many times. The smells can be the scent of flowers, perfume smell, coffee smell, smell of a fried dish, fruit smell, smell of sourness, spicy smell, stinking smell, fishy smell, smoke and fire smells and ammunition smell, and can be also the special made smells. The smell storage space should be numbered and the containers containing different smells should be disposed in according to the numbers. The program card 42 transmits the smell signal to the control circuit board 44 which controls the smell containers 31 to 33 which have different numbers to emit the smells through electromagnetic valves 37 to 39. The smells are composed of compressed gas so that the smells can be emitted out of the smell containers 31 to 33 with enough pressure at the time of the smell emission and enter into the smell room 41 through the spacing baffle 40 to be blown into the air by the fan 27.

The control circuit board 44 is a control circuit or a control unit that carries a CPU. Its input port receives the control signal and its output port is connected respectively to the control end of each control valve 37 to 39. The program card 42 is a unit that transmits the control signal. This unit comprises a smell signal program or a circuit in which a same type of smell corresponds synchronously to the video and audio contents of the television programs, movies, electronic games and computer software. The smell signal program or circuit is connected to the control circuit board 44 through a connection port. As shown in Drawing 2, there is a card plugging jack 25 on the synchronized electronic smell emission box, in which the corresponding program card 42 is plugged for each different program. Different smell containers should be used according to the contents in different television programs, movies, electronic games and computer software. For example, smell container No. 1 is Type A perfume smell. Smell container No. 2 is Type B perfume smell. Smell container No. 3 is jasmine smell and smell container No. 4 is coffee smell. Users can buy the standard smell containers in the market and dispose them at the designated storage space according to the numbers. Certainly, the smell emission box is also a product which can be purchased in the market, especially the uniform standard size of the smell container storage space should be set up. In addition, the smell container emission box can also have some spare disposition spaces in where the smell containers that contain special smells can be put.

The smell emission time, smell type, intensity and the duration of the smell emission are programmed corresponding to the contents in the television programs, movies, electronic games and computer software. At the beginning of the programs, a signal of starting the smell emission should be displayed briefly on the corner of the screen. At this time, the user should turn on the switch immediately or use the remote controller to start the synchronized electronic smell emission box in time so that the emitted smell can match the contents of the picture.

As shown in Drawing 3, when the synchronized electronic smell emission box starts working, digital-digital program card 42 transmits the smell signal separately to the control circuit board 44 which uses the smell signal to separately control the electromagnetic valves 37, 38 and 39 and open the valves and the smells are emitted separately from the container openings 34, 35 and 36 of the smell containers 31, 32 and 33. The signals also control the opening size of the electromagnetic valves in order to control the smell intensity. For example, if jasmine is displayed on the screens of television receivers, movie projectors, electronic game machines and computers 20 minutes after the programs start, the digital-digital program card 42 would transmit a smell signal through the control circuit board 44 to open the smell container No. 3 and emit the jasmine smell (smell is “weak”) for 3 minutes. After 30 minutes, a smell signal would be transmitted to open the smell container No. 4 and emit the coffee smell (smell is “strong”) for 5 minutes. Then the smell is blown into the air by the fan 27 from the smell room 41.

When a program is playing, the smell signals in television receivers, movie projectors, electronic game machines and computers are all transmitted through different types of program cards to program the smell emission type and the time length corresponding to the contents in television programs, movies, electronic games and computer software. The smell signals are saved in the program card 42 in a digital form and are outputted to the control circuit board 44 which then transmits the signals to the corresponding electromagnetic valves which open the corresponding smell containers and emit the corresponding smells. The analog electrical circuit board, digital-analog electrical circuit board or analog-digital electrical circuit board can be also used to save the program information.

The following is the example of the digital circuit on the control circuit board:

The Digital Program Setting of the Smell Type:

| 0000 | 0000 | Natural Air |
|---|---|---|
| No. 1 | 0000 0001 | Starting Electromagnetic Valve of Type A Perfume |
| | | Smell |
| No. 2 | 0000 0010 | Starting Electromagnetic Valve of Type B Perfume |
| | | Smell |
| No. 3 | 0000 0011 | Starting Electromagnetic Valve of Jasmine Smell |
| No. 4 | 0000 0100 | Starting Electromagnetic Valve of Coffee Smell |

The Digital Program Setting of The Smell Intensity

| | | |
|---|---|---|
| 1000 | 0000 | Stopping Electromagnetic Valve |
| 1000 | 0001 | “Weak” Smell (Relatively Small Opening of |
| | | Electromagnetic Valve) |
| 1000 | 0010 | “Relatively Strong” Smell (Relatively Big Opening of |
| | | Electromagnetic Valve) |
| 1000 | 0011 | “Strong” Smell (Big Opening of Electromagnetic Valve) |
| 1000 | 0100 | “Very Strong” Smell (Very Big Opening of |
| | | Electromagnetic Valve) |

The above control circuit board comprises two groups of number settings of smell type and smell intensity. The upper 4 groups of numbers indicate 4 different smell containers (0000 0000 is natural air) and the lower 4 groups of numbers indicate the opening size of the electromagnetic valve (1000 0000 is stopping start-up). The control circuit board programs the digital-analog conversion of the smell type and the digital-analog conversion of the smell intensity. It can also program the digital control directly.

The following is the example of the digital-digital program card programmed for the working principle of the contents in a program:

T1 = 0 (minute)

| | |
|---|---|
| 0000 0000 | Natural Air |
| 1000 0000 | Stopping Electromagnetic Valve |

T2 = 20 (minute)

| | |
|---|---|
| 0000 0011 | Starting Electromagnetic Valve of Jasmine Smell (No. 3) |
| 1000 0001 | “Weak” Smell |

T3 = 23 (minute)

| | |
|---|---|
| 0000 0000 | Natural Air |
| 1000 0000 | Stopping Electromagnetic Valve |

T4 = 30 (minute)

| | |
|---|---|
| 0000 0100 | Starting Electromagnetic Valve of Coffee Smell(No. 4) |
| 1000 0011 | “Strong” Smell |

T5 = 35 (minute)

| | |
|---|---|
| 0000 0000 | Natural Air |
| 1000 0000 | Stopping Electromagnetic Valve |

Contents in one program above are programmed: 20 minutes after a program starts, jasmine is displayed on the screen. The smell is “weak” and the time is 3 minutes. After 30 minutes, the scene of drinking coffee is displayed on the screen. The smell is “strong” and the time is 5 minutes. The smell signals are a group of digital signals saved on the digital-digital program card. 20 minutes after a program starts, the above digital signals will direct the digital circuit on the control circuit board to transmit the corresponding signals to start No. 3 electromagnetic valve of the jasmine smell container and emit “weak” jasmine smell for 3 minutes. After 30 minutes, the No. 4 electromagnetic valve of the coffee smell container starts working and emits “strong” coffee smell for 5 minutes. The smell emission time and smell type correspond to the contents of the picture.

The electromagnetic valves can be used if the smell signals are programmed as no smell intensity. If the smell signals are programmed as having the smell intensity, the special electromagnetic valves should be used, such as servo system electromagnetic valves.

The large synchronized smell emission control device used in the movie theaters can be disposed outside the movie theaters if there is no space to be disposed inside. As shown in Drawing 4, the smells should enter into the movie theaters through the smell suction port 46 at this time and the smell ventilation outlet should be installed at different locations in the movie theaters like the ones of the air conditioning system. Also, the smells can also enter into the movie theaters through the conduit and ventilation outlets of the air conditioning system. In terms of the large synchronized smell emission control device, the smell containers can be disposed outside the emission box.

1. It is a kind of method relating to the synchronized electronic smell emission for television programs, movies and other programs. The characteristic of the synchronized electronic smell emission method is that, according to the corresponding video and audio contents in the television programs, movies, electronic games and computer software, a program card that contains synchronized smell information is pre-produced. The program card operates a group of control valves through control circuit board to open or close a corresponding group and emit a same type of smell from the smell containers which contain different types of smell to correspond synchronously to the video and audio contents in the television programs, movies, electronic games and computer software, and the emitted smells are then blown into the air by the fan.

2. As described in claims 1, the characteristic of the synchronized electronic smell emission method is that the emitted smells enter a smell room first and are then blown into the air by the fan.

3. As described in claims 1, the characteristic of the synchronized electronic smell emission method is that said smells in each smell container are composed of compressed gas of each different type of smell, and the smells are emitted from the smell containers when the control valves are opened.

4. As described in claims 1, or 2, and or 3, the characteristic of the synchronized electronic smell emission method is that the corresponding smell type, emission time, time length and intensity level are shown through the choice of different smell containers and the containers' opening time, time length and the opening size at the time of smell emission. This expression is programmed by the program cards according to the contents required.

5. As described in claims 1, the characteristic of the synchronized electronic smell emission method is that a signal of starting the smell emission is displayed on the screen when the television programs, movies, electronic games and computer software have just started.

6. As described in claims 1, the characteristic of the synchronized electronic smell emission method is that methods of digital-digital control, digital-analog control and analog-digital control are adopted in the whole smell emission control process ranging from the program cards to control valves.

7. As described in claims 1, the characteristic of the synchronized electronic smell emission method is that the emitted smells, in terms of large locations such as the movie theaters, are discharged through the suction port of the air conditioning or ventilation system and that a special discharge port is separately set up.

8. It is a kind of device relating to the synchronized electronic smell emission for television programs, movies and other programs. The characteristic of the synchronized electronic smell emission device is that a group of smell containers are installed in the device and each smell container contains different types of smell. The smell containers are placed separately at each disposition space or at the corresponding locations.
There is a smell room in the device in where the inlet which connects each container opening is made. The control valves are respectively installed at each container opening. The outlet which connects the exterior space is also made in the smell room and a fan is installed at the outlet or in the smell room.
The device is also equipped with a program card and the control circuit board. The control circuit board is either a control circuit or a control unit that carries a CPU.
Its input port receives the control signal and its output port is connected respectively to the control end of each control valve. The program card is a unit that transmits the control signal. This unit comprises a smell signal program or a circuit in which a same type of smell corresponds synchronously to the video and audio contents of the television programs, movies, electronic games and computer software. The unit is connected to the control circuit board through a connection port.

9. As described in claims 8, the characteristic of the synchronized electronic smell emission device is that the said control valves adopt the electromagnetic valves, hydraulic control components, pneumatic components or servo control components.
